(19) United States
(12) Patent Application Publication
(10) Pub. No.: US 2005/0031658 A1
(43) Pub. Date: Feb. 10, 2005

(54) Cosmetic skin treatment process and device for implementing such a process

(75) Inventors: Franck Girier Dufournier, Paris (FR); Anne Liechty, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/902,894

(22) Filed: Aug. 2, 2004

Related U.S. Application Data
(60) Provisional application No. 60/497,557, filed on Aug. 26, 2003.

(30) Foreign Application Priority Data
Aug. 1, 2003 (FR) 03 09573

(51) Int. Cl. A61K 9/14; A61K 7/11

(52) U.S. Cl. 424/401; 424/70.13

(57) ABSTRACT

The present invention relates to a cosmetic treatment process, especially a makeup process, for a surface of skin, mucous membranes or the nails, comprising: the application to the said surface, by spraying, of a cosmetic composition, this composition comprising, in a physiologically acceptable medium, at least one filler for giving the composition a haze index H of greater than or equal to 40% and a hemispheric transmission factor Th of greater than or equal to 70%.

The present invention relates to the cosmetic treatment of the skin, mucous membranes and/or integuments, especially non-fibrous keratin materials.

More particularly, the invention is directed towards proposing a process that is useful especially for at least partially masking skin defects.

It is common to wish to camouflage skin imperfections, in particular of the face or the neck, using foundations.

Conventional foundations generally give large coverage and are useful for matting the complexion and masking skin colour imperfections, such as redness, but are not always suitable for also masking relief defects such as wrinkles.

To camouflage wrinkles and other relief defects, compositions containing “soft-focus” fillers are used. These compositions reduce the perception of the relief of the surface onto which they are applied.

The application of a composition containing soft-focus fillers, manually or using an applicator, onto a surface that has already been made up with a foundation, leads, at least partially, to intimate mixing of the two compositions, which results in a reduction in the efficacy of the soft-focus composition and detracts from the quality of the result.

There is thus a need to benefit from a cosmetic skin treatment process that makes it possible especially to reduce the perception of relief defects, and that also makes it possible, where appropriate, to mask colour defects and to make the complexion matte.

There is moreover a need to further improve the quality of the makeup obtained using a composition containing at least one soft-focus filler.

There is also a need to dissimulate defects without generating a mask effect on, and to maintain a natural appearance for the made-up skin.

The invention is directed towards satisfying all or some of these needs.

According to the first of its aspects, a subject of the present invention is a cosmetic treatment process, especially a makeup process, for a surface of skin, mucous membranes or the nails, this process comprising the application, by spraying onto the said surface, of a soft-focus cosmetic composition, this composition comprising, in a physiologically acceptable medium, at least one filler for giving the composition a haze index H of greater than or equal to 40% and a hemispheric transmission factor Th of greater than or equal to 70%.

The term “spraying” denotes a distribution preferably in the form of a mist of more or less fine particles, especially of droplets.

The application of the composition by spraying can allow a substantially uniform deposition thereof, which contributes towards even better camouflaging the skin relief defects.

The application of the soft-focus composition may be preceded by the application to the surface to be treated of a base composition, which is especially coloured, for example a conventional foundation.

One makeup method according to the invention may thus involve, in a first step, the application by any conventional mode, for instance manually, using an applicator such as a brush or a sponge or by spraying, of a coloured base cosmetic composition. The soft-focus composition according to the invention is then superposed, in a second step, by spraying, onto this first makeup coat.

Spraying advantageously makes it possible to deposit in the form of a thin film, for example of “veil” type, the soft-focus composition onto the surface of the base composition, and thus to prevent any intimate mixing between the two compositions that might be harmful to the development of the desired soft focus.

The invention may thus make it possible to obtain a makeup that is particularly satisfactory in terms of camouflage, whether of coloured imperfections or of relief.

The application of the soft-focus composition may also, as a variant, be simultaneous with the application of the base composition, for example by means of using a spraying device comprising at least two nozzles.

The soft-focus composition may or may not contain at least one colouring agent, especially depending on whether the composition is or is not intended to be superposed onto a coloured base composition.

In the case especially in which the soft-focus composition is applied directly onto the surface to be made up and comprises a colouring agent, the saturation C*of the composition is preferably between 17 and 60 and better still between 20 and 60.

A saturation value C* of greater than 17 allows a sufficient supply of colour to be obtained, whereas a value of less than 60 makes it possible to not detract from the production of the soft focus and can also allow a natural makeup to be obtained.

Measurement of the Hemispheric Transmission Factor Th and Calculation of the Haze Index H

As indicated above, the soft-focus cosmetic composition has a haze index H of greater than or equal to 40%, preferably greater than or equal to 45%, especially 50%, better still 60%, even better greater than or equal to 70%, especially 75% and better still 80%.

The hemispheric transmission factor Th is itself greater than or equal to 70%, especially greater than or equal to 75% and better still greater than or equal to 80%, or even 85%.

The term “haze index H” denotes the magnitude ((Th−Td)/Th). 100, in which Th represents the hemispheric transmission factor and Td represents the direct transmission factor.

When the haze index H is high, the composition gives a large soft focus, allowing defects to be masked by changing the perception of the relief.

The hemispheric transmission factor Th gives information regarding the transparency of the composition. The higher the Th value, the more transparent the composition.

The Th and Td values may be measured using a spectrophotometer and an integration sphere, placed behind the composition that it is desired to characterize.

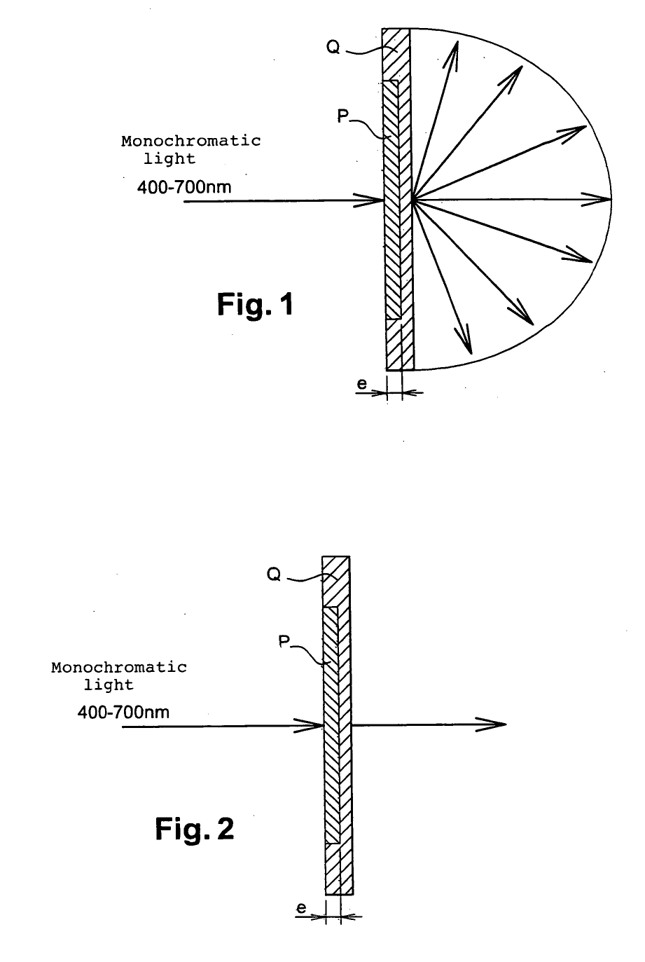

The hemispheric spectral transmission Th(λ) of the composition is defined by the ratio between the intensity of light of wavelength λ received by a sample of composition P and the light intensity restituted by this sample in all directions in a space delimited by a plane, as illustrated in FIG. 1.

The direct spectral transmission Td(λ) of a sample is defined by the ratio between the intensity of light of wavelength λ received by the sample and the light intensity restituted by this sample in the same direction of propagation as the incident light, as illustrated in FIG. 2.

The measurements of the hemispheric spectral transmission Th(λ) and direct spectral transmission Td(λ) may be performed according to the following experimental protocol.

The composition P that it is desired to analyse is spread onto a hollowed-out quartz slide Q, so as to form a layer with a thickness e of 20 μm, and is then placed in an oven for five minutes at 37° C.

The Th(λ) value may be measured using a Varian Cary 300® spectrophotometer and a Labsphere® brand integration sphere placed behind the quartz slide containing the composition. The spectrophotometer is used in diffuse transmission mode and the wavelength λ of the monochromatic incident light ranges from 400 to 700 nm.

The measurement is performed in transmission mode % T, at a sweep speed of 240 nm/minute and in “double reverse” mode.

In a first stage, a calibration is performed by taking a first measurement on the empty quartz slide Q to obtain the maximum value of the transmitted intensity.

Next, the hemispheric spectral transmission Th(λ) is measured with the quartz slide containing the composition P to be analysed.

The Td(λ) value is measured using the same spectrophotometer, used in direct transmission mode and the wavelength λ of the incident monochromatic light ranges from 400 to 700 nm. The machine is set to transmission mode % T, at a sweep speed of 240 nm/minute, in “double” mode. An empty quartz slide is placed in a reference compartment and the quartz slide containing the composition is placed in a measuring compartment, and the Td(λ) is then measured.

The hemispheric transmission factor Th and direct transmission factor Td are calculated by establishing, respectively, the mean of all the Th(λ) or Td(λ) spectral values obtained when the wavelength ranges from 400 to 700 nm.

Saturation C*

The saturation C* may be measured in the CIE L*a*b*C*h colorimetric space, in the following manner.

The composition whose saturation it is desired to determine is placed up to the brim in a galvanized steel crucible having a minimum depth of 2.47 mm.

The crucible is then covered with a 76×26 mm “LMR” H1 bevelled-edge cover slide from the company Labo-Moderne.

The saturation C* is measured using a CM3700d spectrocolorimeter, the illumination and observation system being in d/8° reflectance mode, the measurements being taken in exclusive specular reflection mode.

The UV emission is 100% inclusive.

The zoom position is medium (MAV), the aperture is medium, the configuration is CREIMM, the observation is 10° CIE 1964, and the illuminant is D65.

Spraying Device

The spraying of the soft-focus composition may be performed by any means known to those skilled in the art, provided that these means do not affect the soft-focus properties afforded by the composition.

The spraying may take place, for example, using means for pressurizing the composition, for example by subjecting it to the pressure of a propellent gas.

This propellent gas may be packaged with the composition or separately, the composition being contained, for example, in a flexible bag inside a pressurized container.

The composition may also be pressurized by means of a pump, especially a manually operated pump, which may or may not be with precompression.

The composition may also be sprayed by means of a Venturi effect in a spraying device of aerographic type.

Whatever the spraying mode adopted, the composition may be sprayed via one or more spraying nozzles, with or without turbulence channels.

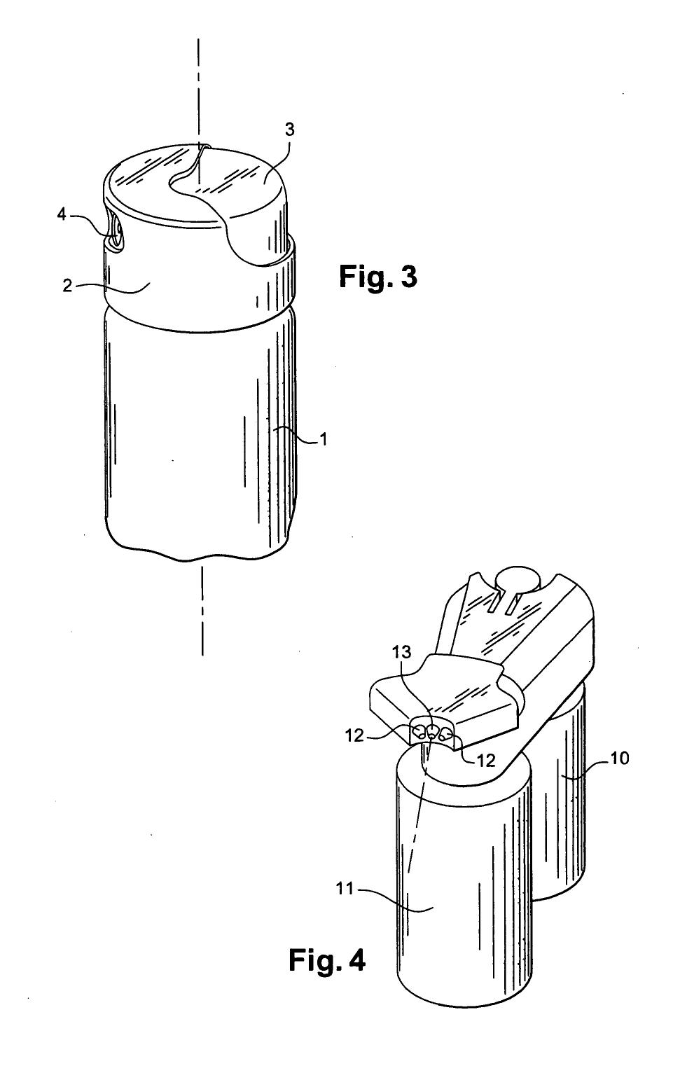

FIGS. 3 and 4 show two examples of spraying devices, among others.

The device in FIG. 3 is, for example, of aerosol type and comprises a pressurized container 1 containing the composition and the propellent gas, a distribution head 2 comprising a push button 3 for actuating a valve, not shown, and a spraying nozzle 4.

FIG. 4 shows another example of a spraying device of aerographic type, comprising a first container 10 containing the carrier gas and a second container 11 containing the composition, the carrier gas 10 being emitted, for example, via two nozzles 12 upstream of a spraying nozzle 13. The negative pressure created by the flow of the carrier gas causes suction in the reservoir 11 of the composition.

Non-limiting examples of spraying devices are described especially in international patent application WO 02/47618.

When the composition is sprayed by means of a propellent gas, this gas may be, for example, a compressed gas such as compressed air or nitrogen, or a liquefiable gas, for instance dimethyl ether, C₃₋₅ alkanes, especially propane, n-butane and isobutane, 1,1-difluoroethane and mixtures of dimethyl ether and of C₃₋₅ alkanes.

The weight ratio of the soft-focus composition/propellent gas may range especially from 0.05 to 50, for example, and in particular from 1.5 to 25.

The spraying may also be performed using a spraying device without a carrier gas or propellent gas, for example an electromechanical or thermal device.

A subject of the invention is also a spraying device comprising a container containing a soft-focus composition as defined above.

This spraying device may comprise means for subjecting the composition to a positive or negative pressure to allow it to be sprayed.

Fillers

The soft-focus fillers that may be used in the soft-focus composition according to the invention may especially comprise or consist of particles with a number-average size of less than or equal to 15 μm, especially less than or equal to 10 μm, in particular less than or equal to 7.5 μm or even less than or equal to 5 μm, for example between 1 μm and 5 μM.

The term “number-average size” denotes the dimension given by the statistical particle size distribution to half the population, known as the D50.

These particles may be of any shape and in particular may be spherical or non-spherical.

The soft-focus fillers according to the invention may be of any chemical nature, provided that they are compatible with cosmetic use and that they do not affect the expected properties of the composition.

They may thus be chosen from silica and silicate powders, especially alumina powders, powders of polymethyl methacrylate (PMMA) type, talc, silica/TiO₂ or silica/zinc oxide composites, polyethylene powders, starch powders, polyamide powders, styrene/acrylic copolymer powders and silicone elastomers, and mixtures thereof.

Among the soft-focus fillers that are particularly suitable for the invention, mention may be made of the talc with a number-average size of less than or equal to 3 microns, for example talc with a number-average size of 1.8 microns and especially the product sold under the trade name Talc P3® by the company Nippon Talc, Nylon® 12 powder, especially the product sold under the name Orgasol 2002 Extra D Nat Cos® by the company Atochem, silica particles surface-treated with a mineral wax 1% to 2% (INCI name: hydrated silica (and) paraffin), such as the products sold by the company Degussa, amorphous silica microspheres, such as the products sold under the name Sunsphere, for example of reference H-53 by the company Asahi Glass, and silica microbeads, such as those sold under the name SB-700® or SB-150® by the company Miyoshi, this list not being limiting.

The soft-focus filler will preferably have a refractive index that is different from that of the medium in which it is present.

The soft-focus filler may be substantially colourless in the composition.

The soft-focus filler may be present in the soft-focus cosmetic composition in a content ranging from 0.1% to 20% by weight and in particular ranging from 1% to 12% by weight, especially between 5% and 10%, for example about 8%, relative to the total weight of the composition.

Colouring Agent

At least one from among the soft-focus composition and the base composition may comprise at least one colouring agent.

The soft-focus composition according to the invention may or may not comprise a colouring agent.

The composition may comprise a colouring agent when it is intended to be applied directly to naked skin, for example.

For the purposes of the present invention, the term “colouring agent” denotes any pigment or dye or mixture of pigments and/or dyes capable of giving the composition sufficient colour to allow it to have the required saturation C*, while at the same time giving the desired haze index H and hemispheric transmission factor Th values.

The colouring agent may especially comprise or consist of particles of at least one pigment. The pigment particles may have undergone, where appropriate, a treatment whose object is to increase the stability of the colour and to facilitate their incorporation into the composition. In particular, pigment particles treated so as to make them hydrophobic will be more readily dispersible in an oily phase, for example.

The pigment particles may have various shapes, especially a substantially spherical or flattened shape.

The pigment particles may have a multilayer structure, and especially a transparent core, for example made of silica.

Such pigment particles with a transparent core can prevent the composition from being excessively opacified, which is favourable for obtaining desired H and Th values.

The pigment may be a non-interference pigment, and may be non-fluorescent.

In the case especially of a pigment whose particles have a relatively transparent multilayer structure, the proportion of pigment may be relatively high, for example greater than or equal to 1% by weight, especially between 1.5% and 10% by weight and better still between 2% and 8% by weight relative to the total weight of the composition. The proportion may be, for example, between 2% and 5%, for example about 3%.

The higher the transparency of the pigment, the greater may be the amount of pigment, in principle, without causing the composition to lose the desired soft focus and hemispheric transmission factor.

The transparency of the pigment may be quantified by its contrast ratio, defined later, this ratio being, for example, between 15 and 65 and better still less than or equal to 50. The contrast ratio may especially be less than or equal to 45, better still less than or equal to 40, or even 35.

The colouring agent may comprise a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The colouring agent may also comprise a pigment having a structure that may be, for example, of silica microsphere type containing iron oxide. An example of a pigment having this structure and a relatively low contrast ratio is the product sold by the company Miyoshi under the reference PC Ball PC-LL-100 P, this pigment consisting of silica microspheres containing yellow iron oxide.

Relatively low contrast ratio values, combined with the haze index and hemispheric transmission factor values, make it possible to obtain particularly satisfactory results in terms of joint supply of colour and coverage, while at the same time maintaining a high soft focus, allowing imperfections to be masked.

In the case of a mixture of pigments, the proportions of these pigments may be adjusted as a function of their contrast ratio.

The composition may be free of pigments with a contrast ratio of greater than 60.

As a variant, the composition may also comprise particles of at least one pigment that is relatively opaque, in particular which has a contrast ratio of greater than 60, with the proviso that the amount introduced into the composition does not prevent the desired H and Th values from being obtained.

The composition may thus comprise, for example, conventional pigments having a relatively high contrast ratio, for example greater than or equal to 40, for example pigments of the iron oxide and/or titanium dioxide type.

The composition preferably comprises less than 2% by weight, better still less than 1.5%, even better still less than 1% and even better still less than 0.75%, for example between 0.25% and 0.75%, for example about 0.5%, of such pigments, relative to the total weight of the composition.

The colouring agent may also be chosen from dyes, especially water-soluble or liposoluble dyes, or other dyestuffs, provided that their incorporation into the composition does not prevent the desired haze index H and hemispheric transmission factor Th from being obtained. The colouring agent may thus comprise a water-soluble dye, for instance caramel.

The colouring agent may have a colour corresponding to that of the skin onto which it is intended to be applied, especially comprising at least one brown, yellow or red pigment.

The colouring agent may be free of nacres.

When the soft-focus composition is sprayed onto a coloured base composition, the soft-focus composition may contain, where appropriate, any colouring agent provided that it does not affect, in particular, the desired H and Th values.

This colouring agent may be chosen from any conventional dyestuff used.

Measurement of the Contrast Ratio

The contrast ratio is defined especially in international patent application WO 98/52534.

To calculate the contrast ratio, the process is performed as follows.

A mixture formed from 5% by weight, relative to the total weight, of the colouring agent to be studied and, for the remainder, from the reference emulsion below is applied to a black opaque support and to a white opaque support, as a film 50 μm thick. This film is dried for 24 hours at 25° C. ±1° C. under a pressure of 1 atmosphere.

The reference emulsion has the formulation:

| | % by weight |
|---|---|
| Water | 45.83 |
| Methyl p-hydroxybenzoate | 0.45 |
| Chlorphenesin | 0.34 |
| Disodium EDTA | 0.11 |
| Glycerol | 5.62 |
| PEG-8 | 2.25 |
| PEG-20 | 1.12 |
| Magnesium aluminium silicate | 0.9 |
| Sodium lauroyl sarcosinate | 1.68 |
| Titanium dioxide (and) alumina (and) glycerol (and) silica | 3.37 |
| Triethanolamine | 1.35 |
| Stearic acid | 2.7 |
| Glyceryl stearate | 2.02 |
| Butyl p-hydroxybenzoate | 0.17 |
| Isononyl isononanoate | 8.99 |
| Cyclohexasiloxane | 6.57 |
| Dimethicone | 10.28 |
| BIS-PEG-15 methyl ether dimethicone | 2.25 |
| Talc | 1.12 |
| Kaolin | 1.12 |
| Polymethyl methacrylate | 1.69 |

Using a colorimeter, for example a Minolta CR-200 machine, in illuminant D65 mode with a viewing angle of 0°, the tristimulus value Y of the composition is measured at three different points on the black support and at three different points on the white support.

The contrast ratio corresponds to the mean of the three Y values measured on the black support, divided by the mean of the three Y values measured on the white support, and multiplied by 100.

The higher and closer to 100% the contrast ratio, the more opaque the colouring agent. The lower the contrast ratio, the more transparent the colouring agent.

Physiologically Acceptable Medium

The soft-focus composition comprises a physiologically acceptable medium.

The term “physiologically acceptable medium” denotes a non-toxic medium that may be applied to the skin, especially the human face and/or neck, lips or nails.

This physiologically acceptable medium may comprise, where appropriate, a liquid fatty phase, this fatty phase possibly comprising at least one oil chosen from volatile oils and non-volatile oils, and mixtures thereof.

Volatile Oil

The liquid fatty phase of the soft-focus composition may comprise at least one volatile oil.

The term “volatile oil” means any non-aqueous medium capable of evaporating on contact with the skin in less than one hour, at room temperature (25° C.) and atmospheric pressure (760 mmHg). The volatile cosmetic oil(s), which is (are) liquid at room temperature, especially has (have) a vapour pressure, measured at room temperature and atmospheric pressure, ranging from 10⁻³ to 300 mmHg (0.266 Pa to 40 000 Pa), preferably from 0.02 to 300 mmHg (2.66 Pa to 40 000 Pa) and better still ranging from 0.1 to 90 mmHg (13 Pa to 12 000 Pa).

These volatile oils may be hydrocarbon-based oils, silicone oils, optionally comprising alkyl or alkoxy groups that are pendent or at the end of a silicone chain, and fluoro oils, or a mixture thereof.

In particular, the volatile oils may be cosmetic oils chosen from oils with no flash point, oils with a flash point ranging from 40° C. to 100° C., and mixtures thereof, in order to facilitate their use. In addition, they advantageously have a boiling point at atmospheric pressure of less than 220° C. and especially less than 210° C., in particular ranging from 110 to 210° C. These volatile oils may, for example, not be monoalcohols containing at least seven carbon atoms.

As volatile oils that may be used, mention may be made of linear or cyclic silicone oils with a viscosity, at room temperature, of less than 8 cSt and especially containing from 2 to 7 silicon atoms, these silicones optionally comprising alkyl or alkoxy groups containing from 1 to 10 carbon atoms.

As volatile silicone oils that may be used, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As other volatile oils that may be used, mention may also be made of hydrocarbon-based volatile oils containing from 8 to 16 carbon atoms and mixtures thereof, and especially branched C₈-C₁₆ alkanes, for instance C₈-C₁₆ isoalkanes (also known as isoparaffins), isododecane, isodecane and isohexadecane, for example the oils sold under the trade names Isopar® or Permethyl®, and branched C₈-C₁₆ esters, for instance isohexyl neopentanoate, and mixtures thereof.

Isododecane (Permethyl 99 A®), C₈-C₁₆ isoparaffins, for instance Isopar L, E, G or H®, mixtures thereof, optionally combined with decametbyltetrasiloxane or with cyclopentasiloxane, or mixtures thereof, are used in particular.

Volatile fluoro oils may also be used.

The volatile oils may be present in the soft-focus composition in a content ranging, for example, from 5% to 97.5% by weight and especially in a content ranging from 20% to 75% by weight relative to the total weight of the composition.

Non-Volatile Oil

The soft-focus composition may also comprise a non-volatile fatty fraction. This non-volatile fatty fraction may comprise at least one non-volatile oil.

The term “non-volatile oil” means a fatty substance that is liquid at room temperature (20° C.) and that does not evaporate at that temperature.

Among the non-volatile oils that may be mentioned are:
poly(C₁-C₂₀)alkylsiloxanes and especially those containing trimethylsilyl end groups, preferably those with a viscosity of less than 0.06 m²/s, among which mention may be made of linear polydimethylsiloxanes and alkylmethylpolysiloxanes such as cetyldimethicone (CTFA name),
silicones modified with optionally fluorinated aliphatic and/or aromatic groups, or with functional groups such as hydroxyl, thiol and/or amine groups,
phenylsilicone oils, especially phenyl trimethicones,
oils of plant or mineral origin, such as liquid paraffin, liquid petroleum jelly, perhydrosqualene, lanolin, apricot oil, wheatgerm oil, sweet almond oil, beauty-leaf oil, sesame seed oil, macadamia oil, grapeseed oil, rapeseed oil, coconut oil, groundnut oil, sunflower oil, palm oil, castor oil, avocado oil, jojoba oil, olive oil or cereal germ oil and the liquid fraction of shea butter; fatty acid esters of polyols, in particular liquid triglycerides, isopropyl myristate, Miglyol oil, isostearyl neopentanoate, 2-ethylhexyl palmitate, castor oil, tributyl acetyl citrate; alcohols, in particular 2-octyldodecanol; acetylglycerides; octanoates, decanoates or ricinoleates of alcohols or of polyalcohols; fatty acid triglycerides; glycerides, and fluoro and perfluoro oils,
amide compounds, in particular those described in patent application PCT/FR98/01077, for instance N-neopentanoyl-2-octyldodecylamine, N-neopentanoyl-2-butyloctylamine, N-(3,5,5-trimethylhexanoyl)-2-octyldodecylamine or N-(3,5,5-trimethylhexanoyl)-2-butyloctylamine, and
mixtures thereof.

In particular, the soft-focus composition may comprise one or more oils chosen from sesame oil, lanolin, isopropyl myristate, Miglyol oil, isostearyl neopentanoate, 2-ethylhexyl palmitate, castor oil, acetyl tributyl citrate, 2-octyldodecanol, N-pentanoyl-2-octyldodecylamine and polydimethylsiloxanes, and/or mixtures thereof.

The non-volatile fatty fraction of the soft-focus composition may also comprise a non-liquid fatty phase and in particular at least one fatty substance chosen from waxes, gums, resins and/or pasty fatty substances of animal, plant, mineral or synthetic origin, and mixtures thereof. The content of non-liquid fatty phase in the soft-focus composition is adjusted such that it can be applied by spraying.

The non-volatile fatty fraction of the soft-focus composition may be present in a content ranging from 1% to 85% by weight and especially in a content ranging from 1% to 30% by weight relative to the total weight of the composition.

The soft-focus composition may also comprise an aqueous phase. This aqueous phase may consist essentially of water. However, it may also comprise a mixture of water and of water-miscible organic solvent (water miscibility of greater than 50% by weight at 25° C.), for instance lower monoalcohols containing from 1 to 5 carbon atoms, such as ethanol and isopropanol, glycols containing from 2 to 8 carbon atoms, such as propylene glycol, ethylene glycol, 1,3-butylene glycol and dipropylene glycol, C₃-C₄ ketones and C₂-C₄ aldehydes.

This aqueous phase (water and optionally the water-miscible organic solvent) may be present in the soft-focus composition in a content ranging from 1% to 95% by weight, especially ranging from 3% to 80% by weight and in particular ranging from 5% to 60% by weight relative to the total weight of the soft-focus composition.

The soft-focus composition may also comprise a film-forming polymer as defined hereinbelow, this film-forming polymer being used under conditions that allow spraying.

In general, the soft-focus composition may also contain adjuvants that are common in cosmetics, such as emulsifiers, hydrophilic or lipophilic gelling agents, hydrophilic or lipophilic active agents, preserving agents, antioxidants, solvents, fragrances, physical sunblocks and chemical sunscreens, odour absorbers and pH regulators. Examples of such adjuvants are given later.

The soft-focus composition may also contain a surfactant, especially one of those mentioned later as being able to form part of the formulation of the base composition.

The fluid consistency of the soft-focus composition is liable to vary according to the nature of the spraying device used and, in the particular case of a device of aerosol type, according to the nature of the propellant.

The soft-focus composition will advantageously be in the form of an emulsion, for example obtained by dispersing a fatty phase in an aqueous phase (O/W) or conversely (W/O), a triple emulsion (W/O/W or O/W/O), a vesicular dispersion of ionic (liposomes or oleosomes) and/or nonionic type (niosomes) and/or a dispersion of nanocapsules or nanospheres.

In the case of an emulsion, the proportion of fatty phase in the composition under consideration may range, for example, from 5% to 80% by weight and especially from 5% to 50% by weight relative to the total weight of the composition.

The soft-focus composition may also be in the form of a sprayable gel, especially a thixotropic gel or a gel that is soluble in the propellent gas, for example.

Base Composition

In the case where it is applied, the base composition also comprises a physiologically acceptable medium.

This physiologically acceptable medium is generally adapted to the nature of the support onto which the composition is to be applied and also to the appearance in which the composition is intended to be packaged.

Thus, the base composition may be more or less fluid and may have the appearance of a coloured or uncoloured cream, an ointment, a milk, a lotion, a serum, a paste, a mousse or a powder. It may also be in solid form, in particular in the form of a stick or a dish, or may have been deposited by coating onto a sheet of paper.

The base composition may be in anhydrous form, emulsion form or gel form.

The base composition may be in any galenical form normally used in cosmetics, and may especially be in the form of an optionally gelled oily solution, an emulsion obtained by dispersing a fatty phase in an aqueous phase (O/W) or conversely (W/O), a triple emulsion (W/O/W or O/W/O), an aqueous gel, a vesicular dispersion of ionic type (liposomes or oleosomes) and/or nonionic type (niosomes) and/or a dispersion of nanocapsules or nanospheres.

In the case of an emulsion, the proportion of fatty phase of the composition under consideration may range, for example, from 5% to 80% by weight and especially from 5% to 50% by weight relative to the total weight of the composition.

The base composition may comprise at least one aqueous phase.

The aqueous phase may consist essentially of water. However, it may also comprise a mixture of water and of water-miscible organic solvent (miscible in water to greater than 50% by weight at 25° C.), for instance lower monoalcohols containing from 1 to 5 carbon atoms, such as ethanol, isopropanol, glycols containing from 2 to 8 carbon atoms, such as propylene glycol, ethylene glycol, 1,3-butylene glycol, dipropylene glycol, C₃-C₄ ketones and C₂-C₄ aldehydes.

This aqueous phase (water and optionally the water-miscible organic solvent) may be present in the base composition in a content ranging from 1% to 95% by weight, especially ranging from 3% to 80% by weight, and in particular ranging from 5% to 60% by weight, relative to the total weight of the base composition.

The base composition may also comprise a fatty phase.

In particular, the fatty phase of the base composition may comprise at least one oil chosen especially from:
hydrocarbon-based oils of animal origin, such as perhydrosqualene;
hydrocarbon-based oils of plant origin, such as liquid triglycerides of fatty acids containing from 4 to 10 carbon atoms and the liquid fraction of karite butter;
synthetic esters and synthetic ethers, especially of fatty acids, for instance oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, and hydrogenated polyisobutene such as parleam oil;
fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils, for instance those described in document JP-A-2 295 912;
silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates and polymethylphenylsiloxanes; and
mixtures thereof.

The base composition may also comprise at least one wax, at least one gum and/or at least one pasty fatty substance, of plant, animal, mineral or synthetic origin, of silicone or non-silicone nature.

The waxes may be hydrocarbon-based waxes, silicone waxes and/or fluoro waxes, optionally comprising ester or hydroxyl functions. They are especially of natural origin.

The wax may represent from 0.01% to 10% by weight and especially from 0.1% to 5% by weight relative to the total weight of the base composition. According to one embodiment, the composition may be wax-free.

The base composition may also comprise at least one film-forming polymer.

According to the present invention, the term “film-forming polymer” means a polymer that is capable of forming, by itself or in the presence of an auxiliary film-forming agent, a continuous film that adheres to the skin.

A film-forming polymer capable of forming a hydrophobic film, i.e. a polymer whose film has a solubility in water at 25° C. of less than 1% by weight, is advantageously used.

The film-forming polymer may especially be at least one polymer chosen from the group comprising:
water-soluble film-forming polymers,
aqueous dispersions of water-dispersible film-forming polymer particles, also known as “latices”: in this case, the composition of foundation type must comprise an aqueous phase,
liposoluble film-forming polymers,
lipodispersible film-forming polymers in the form of non-aqueous dispersions of polymer particles, preferably dispersions of polymer particles surface-stabilized, where appropriate, with at least one stabilizer, in one or more silicone oils and/or hydrocarbon-based oils; these non-aqueous dispersions are also known as “NADs”, and
mixtures thereof.

Aqueous dispersions of film-forming polymers that may be used according to the invention include the acrylic dispersions sold under the names Neocryl XK-90®, Neocryl A-1070®, Neocryl A-1090®, Neocryl BT-62®, Neocryl A-1079® (and Neocryl A-523® by the company Avecia-Neoresins, Dow Latex 432® by the company Dow Chemical, Daitosol 5000 AD® by the company Daito Kasey Kogyo; or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer.

Aqueous dispersions of film-forming polymer that may also be mentioned include the polymer dispersions resulting from the free-radical polymerization of one or more free-radical monomers inside and/or partially at the surface of pre-existing particles of at least one polymer chosen from the group consisting of polyurethanes, polyureas, polyesters, polyesteramides and/or alkyd polymers. These polymers are generally referred to as hybrid polymers.

As examples of water-soluble film-forming polymers that may be used according to the invention, mention may be made of proteins, for instance proteins of plant origin, anionic, cationic, amphoteric or nonionic chitin or chitosan polymers, cellulose polymers, acrylic polymers or copolymers, vinyl polymers and polymers of natural origin, which are optionally modified, and mixtures thereof.

Dispersions of film-forming polymer in the liquid fatty phase, in the presence of stabilizers, are described especially in documents EP-A-749 746, EP-A-923 928 and EP-A-930 060, the content of which is incorporated into the present patent application by reference.

Examples of liposoluble polymers which may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

The film-forming polymer may be present in the composition in a solids content ranging from 0.01% to 20% by weight and especially from 0.5% to 10% by weight relative to the total weight of the composition.

The base composition may also contain adjuvants that are common in cosmetics, such as emulsifiers, hydrophilic or lipophilic gelling agents, dyestuffs, hydrophilic or lipophilic active agents, preserving agents, antioxidants, solvents, fragrances, fillers, physical sunblocks and chemical sunscreens, odour absorbers and pH regulators.

The amounts of these various adjuvants are those conventionally used in the field under consideration, for example from 0.01% to 20% of the total weight of the composition. Depending on their nature, these adjuvants may be introduced into the fatty phase or into the aqueous phase.

In any case, these adjuvants, and the proportions thereof, will be chosen so as not to harm the desired properties according to the invention.

The base composition may also comprise at least one surfactant.

These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions of surfactants, in particular pp. 347-377 of this reference, for the anionic and nonionic surfactants.

Among the surfactants that may especially be used in the base composition, mention may be made especially of:
nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated C₁-C₆ alkyl glucose fatty esters, and mixtures thereof,
anionic surfactants: C₁₆-C₃₀ fatty acids neutralized with amines, aqueous ammonia or alkaline salts, and mixtures thereof.

As emulsifiers and co-emulsifiers that may be used, mention may be made, for example, of O/W emulsifiers such as fatty acid esters of polyethylene glycol; especially PEG-100 stearate, and fatty acid esters of glycerol, such as glyceryl stearate, and also W/O emulsifiers available, for example, under the trade names Abil WE09, Abil EM 90, Abil EM97 from the company Degussa Goldschmidt, or the mixture of acetyl ethylene glycol stearate and of glyceryl tristearate sold by the company Guardian under the trade name Unitwix.

As hydrophilic gelling agents that may be used, mention may be made in particular of carboxyvinyl polymers (carbomer), acrylic copolymers such as acrylate/alkylacrylate copolymers, polyacrylamides, polysaccharides, natural gums and clays, and lipophilic gelling agents that may be mentioned include modified clays, for instance bentones, metal salts of fatty acids, hydrophobic silica and polyethylenes.

As dyestuffs that may be used in particular in the base composition, mention may be made of lipophilic dyes, hydrophilic dyes, pigments and nacres usually used in cosmetic or dermatological compositions, and mixtures thereof.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow.

The pigments may be white or coloured, mineral and/or organic, and coated or uncoated. Among the mineral pigments that may be mentioned are titanium dioxide, optionally surface-treated, zirconium oxide or cerium oxide, and also iron oxide or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacreous pigments may be chosen from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the abovementioned type, and nacreous pigments based on bismuth oxychloride.

The pigments may have undergone a surface treatment.

Among the pigments that may be used in the base composition, mention may also be made, of goniochromatic pigments.

As fillers that may be used in the base composition, mention may be made, besides the fillers described above in relation to the soft-focus composition of pigments, starch crosslinked with octenylsuccinic anhydride sold by the company National Starch under the name Dry Flo Plus (28-1160); polyamide particles and especially those sold under the name Orgasol by the company Atochem; microspheres based on acrylic copolymers, such as those made of ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; polymethacrylate-type powders (PMMA), silicone elastomers and silica powders of the Sunsphere type, silicone resin microbeads such as those sold under the name Tospearl by the company Toshiba Silicone; and mixtures thereof.

These fillers may be present in amounts ranging from 0.01% to 20% by weight and preferably from 1% to 10% by weight, relative to the total weight of the base composition.

Fillers that are advantageous for use in the present invention are silica, mica and titanium dioxide.

Active Agents

As active agents that are common in cosmetics or dermatology, which may be used in the base composition and/or in the soft-focus composition, mention may be made in particular of any active agent known for its activity on ageing of the skin, for instance keratolytic agents or prodesquamating agents, for example α-hydroxy acids, β-hydroxy acids, α-keto acids, β-keto acids, retinoids and esters thereof, retinal, and retinoic acid and its derivatives. Mention may also be made of venotonic plant extracts such as extracts of ruscus and/or of common horse chestnut; xanthine bases such as caffeine; vitamins, for instance vitamins A, B3, PP, B5, E, K1 and/or C and derivatives of these vitamins and especially esters thereof; free-radical scavengers; sunscreens; moisturizers, for instance polyols; ceramides; DHEA and its derivatives; coenzyme Q10; bleaching agents and depigmenting agents, for instance kojic acid, extracts of skullcap, of mulberry, of liquorice and/or of camomile; para-aminophenol derivatives, arbutin and derivatives thereof, and mixtures thereof.

For use in the cosmetic treatment of greasy or combination skin, the base composition and/or the soft-focus composition may contain at least one active agent chosen from: vitamins B3 and B5; zinc salts, in particular zinc oxide and zinc gluconate; salicylic acid and its derivatives, such as 5-n-octanoylsalicylic acid; triclosan; capryloyl-glycine; an extract of clove; octopirox; hexamidine; and azelaic acid and its derivatives.

UVA and/or UVB screening agents chosen from organic screening agents and mineral screening agents that are optionally coated to make them hydrophobic may also be introduced into the base composition and/or into the soft-focus composition.

Makeup Kit

According to another of its aspects, the present invention also relates to a makeup kit. This makeup kit comprises at least one base composition and a soft-focus composition according to the invention, which is different from the base composition.

This makeup kit may also comprise means for applying the base composition to the skin, for example.

The base composition and the soft-focus composition, forming this makeup kit, may or may not be packaged in separate packaging articles.

The formulation examples presented below are given as non-limiting illustrations of the invention, the percentages being expressed on a weight basis relative to the total weight of the composition.

### Example of a Base Composition Consisting of a Foundation

| | % |
|---|---|
| | by weight |
| 1,3-Butylene glycol | 10.00 |
| Hectorite modified with distearyldimethylammonium chloride | 1.60 |
| (sold under the name Bentone 38 V by Elementis) | |
| Preserving agents | 0.90 |
| Cyclopentadimethylsiloxane | 11.36 |
| Isostearyl neopentanoate | 0.50 |
| Sodium chloride | 0.70 |
| Isododecane | 13.00 |
| Cyclohexadimethylsiloxane | 8.00 |
| Polydimethylsiloxane (DC 200 Fluid 5 cSt | 2.00 |
| sold by Dow Corning) | |
| Cetyl dimethicone copolyol (sold under the name Abil EM 90 | 0.80 |
| by Goldschmidt) | |
| Polyglyceryl isostearate | 0.60 |
| Isoeicosane | 2.00 |
| Hexyl laurate | 0.60 |
| Hollow microspheres of polymethyl methacrylate (sold under | 2.00 |
| the name Covabead LH 85 by Wackherr) | |
| Polymethyl methacrylate powder (sold under the name | 2.00 |
| Jurymer MB1 by Nihon Junyaku) | |
| Oxyethylenated polydimethylsiloxane (sold under the name | 4.48 |
| KF-6017 from Shin-Etsu) | |
| Nacre | 1.00 |
| Yellow iron oxide coated with perfluoroalkyl phosphate as a | 2.40 |
| dispersion in decamethylcyclopentasiloxane/dimethicone | |
| copolyol (sold under the name FA50DYF by Kobo) | |
| Brown iron oxide coated with perfluoroalkyl phosphate as a | 0.93 |
| dispersion in cyclomethicone/dimethyl polysiloxane copolyol | |
| (sold under the name FA50DRF by Kobo) | |
| Black iron oxide coated with perfluoroalkyl phosphate as a | 0.44 |
| dispersion in decamethylcyclopentasiloxane/dimethicone | |
| copolyol (sold under the name FA65DBF by Kobo) | |
| Alumina-treated titanium oxide coated with perfluoroalkyl | 9.54 |
| phosphate in decamethylcyclopentasiloxane/dimethicone | |
| copolyol (sold under the name FA65DF by Kobo) | |
| Water | qs 100.00 |

### Example of an Uncoloured Soft-Focus Composition Intended to be Sprayed Onto the Above Base Composition

| | % by weight |
|---|---|
| Polyglyceryl-4 isostearate (and) hexyl laurate (and) | 9 |
| cetyl PEG/PPG-10/1 dimethicone | |
| Acetylated glycol stearate | 0.7 |
| Butyl p-hydroxybenzoate | 0.15 |
| 2-Oleamido-1,3-octadecanediol | 0.04 |
| Cyclopentasiloxane (and) disteardimonium hectorite | 8 |
| (and) denatured alcohol | |
| Cyclopentasiloxane | 14.25 |
| Dimethicone | 4 |
| Isododecane | 2.6 |
| Talc P3 from the company Nippon Talc | 8 |
| Water | 47.06 |
| Magnesium sulfate | 0.7 |
| Methyl p-hydroxybenzoate | 0.25 |
| Propylene glycol | 5 |
| Chlorphenesin | 0.25 |

The soft-focus composition is applied onto the base composition using a spraying device of aerographic type.

A substantial reduction in the perception of the skin imperfections may be observed.

### Example of a Coloured Soft-Focus Composition that May be Sprayed onto Naked Skin

| | % by weight |
|---|---|
| Polyglyceryl-4 isostearate (and) hexyl laurate (and) | 9 |
| cetyl PEG/PPG-10/1 dimethicone | |
| Acetylated glycol stearate | 0.7 |
| Butyl p-hydroxybenzoate | 0.15 |
| 2-Oleamido-1,3-octadecanediol | 0.04 |
| Cyclopentasiloxane (and) disteardimonium hectorite (and) | 8 |
| denatured alcohol | |
| Cyclopentasiloxane | 14.25 |
| Dimethicone | 4 |
| Isododecane | 2.6 |
| Coverleaf NS pigment from the company | 3 |
| Chemicals and Catalysts | |
| Talc P3 from the company Nippon Talc | 8 |
| Water | 44.06 |
| Magnesium sulfate | 0.7 |
| Methyl p-hydroxybenzoate | 0.25 |
| Propylene glycol | 5 |
| Chlorphenesin | 0.25 |

The composition is applied using a spraying device of aerographic type.

A supply of colour and a reduction in the perception of the relief defects may be observed.

### Example of Another Coloured Soft-Focus Composition that May be Sprayed Onto Naked Skin

| | % by weight |
|---|---|
| Polyglyceryl-4 isostearate (and) hexyl laurate (and) | 9 |
| cetyl PEG/PPG-10/1 dimethicone | |
| Acetylated glycol stearate | 0.7 |
| Butyl p-hydroxybenzoate | 0.15 |
| 2-Oleamido-1,3-octadecanediol | 0.04 |
| Cyclopentasiloxane (and) disteardimonium hectorite | 8 |
| (and) denatured alcohol | |
| Cyclopentasiloxane | 14.25 |
| Dimethicone | 4 |
| Isododecane | 2.6 |
| Iron oxides (and) disodium stearoyl glutamate (and) | 0.18 |
| aluminium hydroxide | |
| Titanium dioxide (and) disodium stearoyl glutamate (and) | 0.32 |
| aluminium hydroxide | |
| Talc P3 from the company Nippon Talc | 8 |
| Water | 46.56 |
| Magnesium sulfate | 0.7 |
| Methyl p-hydroxybenzoate | 0.25 |
| Propylene glycol | 5 |
| Chlorphenesin | 0.25 |

The composition is applied as previously using a spraying device of aerographic type. A reduction in the visibility of the colour imperfections and relief imperfections of the skin is again observed.

Throughout the description, including the claims, the term “comprising a” should be understood as being synonymous with “comprising at least one”, unless otherwise mentioned.

1. Cosmetic treatment process, especially a makeup process, for a surface of skin, mucous membranes or the nails, comprising:
the application to the said surface, by spraying, of a cosmetic composition, this composition comprising, in a physiologically acceptable medium, at least one filler for giving the composition a haze index H of greater than or equal to 40% and a hemispheric transmission factor Th of greater than or equal to 70%.

2. Process according to claim 1, characterized in that the composition has a haze index of greater than or equal to 45%, especially greater than or equal to 50%; in particular greater than or equal to 60%, or even greater than or equal to 70%, and better still 80%.

3. Process according to claim 1 or 2, characterized in that the composition has a hemispheric transmission factor Th of greater than or equal to 75%, especially greater than or equal to 80% and in particular greater than or equal to 85%.

4. Process according to any one of the preceding claims, characterized in that the filler consists of particles with a number-average size of less than or equal to 15 μm, especially less than or equal to 10 μm, in particular less than or equal to 7.5 μm, or even less than or equal to 5 μm.

5. Process according to any one of the preceding claims, characterized in that the filler is chosen from silica or silicate powders, especially alumina powders, talc, powders of polymethyl methacrylate (PMMA) type, silica/TiO₂ or silica/zinc oxide composites, polyethylene powders, starch powders, polyamide powders, styrene/acrylic copolymer powders and silicone elastomers, and mixtures thereof.

6. Process according to any one of the preceding claims, characterized in that the filler is chosen from talc particles with an average size of less than or equal to 3 μm, Nylon® 12 powder, silica particles surface-treated with a mineral wax, amorphous silica microspheres and silica microbeads, and mixtures thereof.

7. Process according to any one of the preceding claims, characterized in that the filler is present in a proportion of from 0.1% to 20% by weight and in particular from 1% to 12%, better still from 5% to 10% by weight and especially about 8% by weight, relative to the total weight of the composition.

8. Process according to any one of the preceding claims, characterized in that the composition comprises at least one liquid fatty phase, comprising at least one oil chosen from volatile oils and non-volatile oils, and mixtures thereof.

9. Process according to claim 8, characterized in that the composition comprises at least one volatile oil chosen from linear or cyclic silicone oils with a viscosity at room temperature of less than 8 cSt, volatile hydrocarbon-based oils containing from 8 to 16 carbon atoms and fluoro oils, and mixtures thereof.

10. Process according to claim 8 or 9, characterized in that the composition comprises at least one volatile oil chosen from isododecane and C₈ to C₁₆ isoparaffins, and mixtures thereof

11. Process according to claim 10, characterized in that the composition comprises at least one volatile oil combined with decamethyltetrasiloxane or cyclopentasiloxane, or a mixture thereof.

12. Process according to any one of claims 8 to 10, characterized in that the composition comprises at least one volatile oil in a content ranging from 5% to 97.5% by weight and especially in a content ranging from 20% to 75% by weight relative to the total weight of the composition.

13. Process according to any one of the preceding claims, characterized in that the liquid fatty phase comprises at least one non-volatile oil chosen from poly(C₁-C₂₀)alkylsiloxanes, silicones modified with aliphatic and/or aromatic groups, phenylsilicone oils, oils of plant or mineral origin and amide compounds, and mixtures thereof.

14. Process according to claim 13, characterized in that the non-volatile oil is chosen from sesame oil, lanolin, isopropyl myristate, Miglyol oil, isostearyl neopentanoate, 2-ethylhexyl palmitate, castor oil, acetyl tributyl citrate, 2-octyldodecanol, N-pentanoyl-2-octyldodecylamine and polydimethylsiloxanes, and mixtures thereof.

15. Process according to any one of the preceding claims, characterized in that the composition also comprises a non-liquid fatty phase.

16. Process according to any one of the preceding claims, characterized in that the composition comprises a non-volatile fatty fraction present in a proportion of from 1% to 85% by weight and especially from 1% to 30% by weight relative to the total weight of the composition.

17. Process according to any one of the preceding claims, characterized in that it also comprises the prior application to the surface of at least one base composition that is different from the soft-focus composition, before the step of spraying the soft-focus composition.

18. Process according to claim 17, characterized in that the base composition is coloured.

19. Process according to any one of claims 1 to 16, characterized in that the spraying of the soft-focus composition is performed simultaneously with the spraying of at least one base composition that is different from the said soft-focus composition.

20. Process according to any one of claims 17 to 19, characterized in that at least one from among the soft-focus composition and the base composition comprises at least one colouring agent.

21. Makeup kit comprising at least one cosmetic base composition and a soft-focus focus composition that is different from the base composition, as defined in any one of claims 1 to 20.

22. Spraying device comprising a container containing a composition comprising, in a physiologically acceptable medium, at least one filler for giving the composition a haze index H of greater than or equal to 40% and a hemispheric transmission factor Th of greater than or equal to 70%.

23. Device according to claim 22, characterized in that it comprises means for subjecting the composition to a pressure that allows it to be sprayed.

24. Device according to claim 22, characterized in that it comprises means for subjecting the composition to a negative pressure that allows it to be sprayed.
